(19) United States
(12) Patent Application Publication
(10) Pub. No.: US 2013/0341192 A1
(43) Pub. Date: Dec. 26, 2013

(54) COMPENSATED PATCH-CLAMP AMPLIFIER FOR NANOPORE POLYNUCLEOTIDE SEQUENCING AND OTHER APPLICATIONS

(71) Applicants: William Dunbar, Santa Cruz, CA (US); Jungsuk Kim, Santa Cruz, CA (US); Kenneth Pedrotti, Santa Cruz, CA (US)

(72) Inventors: William Dunbar, Santa Cruz, CA (US); Jungsuk Kim, Santa Cruz, CA (US); Kenneth Pedrotti, Santa Cruz, CA (US)

(73) Assignee: THE REGENTS OF THE UNIVERSITY OF CALIFORNIA, Oakland, CA (US)

(21) Appl. No.: 13/978,477

(22) PCT Filed: Jul. 18, 2012
(86) PCT No.: PCT/US2012/047231

§ 371 Date: Sep. 14, 2013

Related U.S. Application Data
(60) Provisional application No. 61/572,829, filed on Jul. 20, 2011.

(51) Int. Cl. G01N 33/487; H03F 3/45

(52) U.S. Cl. CPC G01N 33/48728; H03F 3/45179
USPC 204/601; 330/260

(57) ABSTRACT

A compensated patch-clamp system for polynucleotide sequencing and other applications.

### RELATIONSHIP TO OTHER APPLICATIONS

To the extent allowed by law this application claims priority to and the benefit of U.S. provisional patent application Ser. No. 61/572,829 filed 20 Jul. 2011, entitled “A SWITCHED VOLTAGE PATCH-CLAMP AMPLIFIER FOR DNA SEQUENCING ON SOLID-STATE NANOPORE”. That application and any publication cited therein are hereby incorporated by reference to the fullest extent allowed by law.

### STATEMENT OF SUPPORT

This invention was made partly using funds from the National Science Foundation, NSF Career grant number ECCS-0845766. The US Federal Government has certain rights to this invention.

### FIELD OF THE INVENTION

The presently disclosed subject matter is directed towards electronic devices and systems suitable for use in DNA sequencers and for detecting and quantifying individual nucleotides in a polynucleotide. More particularly, the present invention relates to compensated patch-clamp amplifiers and their use in DNA sequencing systems and methods and in similar applications.

### BACKGROUND OF THE INVENTION

DNA was first isolated from cells by the Swiss scientist Friedrich Miescher in 1869. In 1944 Deoxyribonucleic Acid was discovered to be a chemical that comprised a tiny genetic encyclopedia in living cells. In 1953 James Watson, an American scientist, and Francis Crick, a British researcher working at the University of Cambridge in England discovered the now-famous “double helix” molecular structure of DNA for which they received a 1962 Nobel Prize.

In nanopore sequencing a DNA strand to be sequenced is passed through an ionic fluid filled sensor having a very small pore while a voltage is induced across the sensor. The resulting sensor current depends on the structure of the DNA strand. By analyzing the sensor current the DNA strand can be sequenced. While the theoretical framework of nanopore sequencing is well understood, prior art nanopore sequencing systems and devices were not fully developed. Nanopore sequencing currents are very small and any realistic nanopore sequencing system requires very high gains. Very high gains tend to create reading instabilities caused by distributed resistances and capacitances as well as internal and external noise.

Despite those problems the promise of nanopore sequencing has motivated the development of electronic devices and systems that can detect and quantify individual nucleotides in a polynucleotide. In practice a nanopore sensor has two chambers, referred to as a cis and a trans chamber. Those chambers are filled with a buffered ionic conducting solution (for example, KCl) and a voltage is applied across the nanopore chambers. As a result, a charged DNA initially placed in the cis chamber starts moving towards the trans side. As it traverses the nanopore, the ionic current momentarily decreases. The ionic current is typically in the range of tens to hundreds of picoAmperes. The resulting electric current depends on the number of ions (the charge/net charge) in the nanopore as well as on the nanopore dimensions. The number and charge of ions can be the result of the DNA nucleotide strand passing through the nanopore (or approaching the nanopore opening). It is by monitoring the resulting current that the DNA nucleotide can be sequenced.

Accurately measuring the ultra-low current variations requires a very specialized amplifier that is referred to herein as a patch-clamp. Practical patch-clamps include an input headstage current-to-voltage converter and a difference amplifier that amplifies the voltage from the headstage. A patch-clamp must meet two very challenging design requirements. First, the input-offset voltage (V_{OS}) of the headstage must be minimized. Even the best high-gain amplifiers available have some V_{OS}. Causes for the V_{OS} include random process mismatches and unavoidable systematic variations. Whatever the V_{OS} is, it is amplified by the difference amplifier. In effect the V_{OS} limits the output dynamic range.

Secondly, patch-clamp input parasitic capacitances have to be reduced to prevent headstage saturation. When a command voltage V_{CMD} is applied to the nanopore sensor to produce operating currents, that voltage is actually applied through a resistance to an inverting input of an op-amp. Thus a command voltage V_{CMD} change is time delayed due to unavoidable stray system capacitances. This causes a transient difference between the inverting input and the non-inverting input which leads to output saturation until the parasitic capacitances are charged and the inverting input once again is equal to V_{CMD}. During this interval, known as the ‘dead-time’ all incoming data is lost. Minimizing V_{OS} and compensating for input parasitic capacitances and resistance are major design problems in nanopore sequencing.

Modern patch-clamps are rather specialized high gain, differential op-amp transimpedance amplifiers that use either resistive or capacitive feedback. FIGS. 1(a) and 1(b) present those two basic patch-clamp architectures. In any event, the basic patch-clamp comprises two components: an amplifier 10 and a compensation system that comprises either a resistor 12, reference the resistive feedback patch-clamp circuit 6 shown in FIG. 1(a), or a capacitor 14 in parallel with a reset switch 16, reference the capacitive feedback patch-clamp circuit 8 shown in FIG. 1(b). In both circuits a command voltage V_{CVM} is applied to the non-inverting input 17 of the amplifier 10 while the potential across a nanopore sensor 302 (see for example FIG. 6) is applied to the inverting input 18.

In FIG. 1(a), the input current Iᵢₙ on the inverting input 18 is amplified in accord with the value of the feedback resistor 12 (R_{f}). The resulting transimpedance gain is simply V_{OUT}=R_{f}×Iᵢₙ. In FIG. 1(b), the capacitive feedback acts as an integrator, and thus the amplifier 10 must in practice be followed by a differentiator.

In theory the basic patch-clamps 6 and 8 are sound. In practice, things go wrong. Transimpedance patch-clamp amplifiers that use resistive feedback, reference FIG. 1(a), suffer from significant time delays following command voltage V_{CMD} changes. Referencing the nanopore sensor 302 shown in FIG. 6, those delays are a result of result of a pole-zero characteristics, the relatively large feedback resistor 12 (see FIG. 1(a)), an unavoidable series resistance R_{S} 303, the nanopore sensor 302 capacitance (C_{N}) 305, and the nanopore sensor 302 resistance (R_{N}) 307. The resistive feedback patch-clamp circuit 6 shown in FIG. 1(a) operates as a non-inverting amplifier with a gain of (1+C_{N}/C_{P}) just after the command voltage V_{CMD} changes. Since C_{N} is always larger than C_{P} the output of the amplifier 10 becomes saturated and data is lost until the amplifier 10 has time to supply sufficient charge to the capacitances to allow a return to normal operation. That ‘dead-time’ is very undesirable.

In the prior art, complicated compensation circuitry has been used to attempt to avoid, shorten, or at least minimize dead-time. Such prior art compensation circuitry not only increased the complexity of the basic patch-clamp but resulted in an increased input capacitance which not only limited the bandwidth of resistive feedback patch-clamp circuits, such as the resistive feedback patch-clamp circuit 6, but usually resulted in output voltage “ringing” in response to a step input.

The capacitive feedback patch-clamp circuit 8 shown in FIG. 1(b) was developed at least in part to avoid the dead-time and system complexity of resistive feedback patch-clamp circuits 6 (see FIG. 1(a)). The capacitive feedback patch-clamp circuit 8 has a wide bandwidth and effectively a unity gain at the instant when the reset switch 16 is closed. By properly timing the closing of the reset switch 16 across the capacitor 14 having a capacitance of C_{f}, a command voltage V_{CMD} change on the non-inverting terminal 17 does not initially affect the output of the amplifier 10 and output saturation is avoided.

Unfortunately, when the reset switch 16 opens, the input capacitance at the inverting input 18 increases by C_{f}×(1+A₀), wherein Aₒ is the gain of the amplifier 10, reference the well-known Miller's theorem. That rather dramatic input capacitance change subsequently restricts the bandwidth of the capacitive feedback patch-clamp circuit 8. Thus using capacitive feedback transimpedance amplifiers makes it very difficult to apply arbitrary command voltage V_{CMD} changes because the reset frequency (f_{RST}) is determined by Iᵢₙ/(C_{f}×ΔV), where ΔV is the voltage difference between the inverting input 18 and the output voltage V_{O}. That frequency is not necessarily synchronized with command voltage V_{CMD} changes.

One solution to the reset frequency-command voltage V_{CMD} change problem is to simply increase the reset frequency (f_{RST}) by decreasing the capacitance C_{f} of the capacitance 14 so that the reset frequency is compatible with the command voltage V_{CMD} changes. This requires multiple capacitors and their proper selection as feedback capacitor 14 capacitances whenever waveforms having different transition periods are applied as the command voltage V_{CMD} changes. The result is a much larger and more complex patch-clamp amplifier.

Prior art compensation of patch-clamp amplifiers used additional amplifiers to estimate series resistance (R_{S}) and parasitic capacitance (C_{P}), a rather complex circuit resulted.

Therefore, a new patch-clamp amplifier circuit that avoids the foregoing and other limitations in the prior art would be desirable. Even more desirable would be new patch-clamp amplifier systems that incorporate compensation tailored to the particular application. Ever more beneficial would be new patch-clamp systems having compensation that can be digitally controlled.

### BRIEF SUMMARY OF THE INVENTION

The principles of the present invention provide for techniques for patch-clamp amplifier circuits that incorporate compensation and that can be tailored to a particular application. The new patch-clamp circuit uses digitally controlled compensation and can be used in a nanopore sequencer for sequencing polynucleotides.

Those principles are incorporated in a patch-clamp circuit having a clock that produces timing signals. The patch-clamp circuit further includes a differential amplifier circuit having a non-inverting input, an inverting input with a parasitic capacitance and an electrode resistance, and an output. A feedback resistor is connected between the output and the inverting input. A reset switch receives the timing signals and in response selectively connects the output to the inverting. A command voltage circuit receives command voltages and timing signals. The command voltage circuit produces stepped command voltages that are applied to the non-inverting input in response to the timing signals. A sensor having an input capacitance and a series resistance is operatively connected to the inverting input. The reset switch closes for a time TR in synchronization with step changes in the stepped command voltages and then opens. The time TR is sufficient to prevent saturation of the differential amplifier circuit during the step changes but without blanking out the stepped voltage. The stepped command voltages are selected to compensate for the series resistance and the electrode resistance so as to produce predetermined voltages across the sensor.

In practice the patch-clamp system uses a nanopore sensor while the differential amplifier circuit can have a current to voltage converter and a difference amplifier. The command voltage circuit may be a sample and hold circuit, a Digital-to-Analog converter or some other type of circuit that produces well defined steps. In practice the output can be applied to an Analog-to-Digital converter that produces an amplified digital version of the current in the sensor. The digital version can be applied to a field programmable array or otherwise input into a computer. Preferably that computer causes the command voltages to be applied to the command voltage circuit.

The principles of the present invention also enable methods of compensating sensors used in patch-clamp systems. Such a method involves connecting a first end of an electrode to the inverting input of a patch-clamp system, connecting the second end of the electrode to ground, and connecting a feedback resistor R_{F} between the inverting input and the output of the patch-clamp system. This enables obtaining a steady state output from the patch-clamp system. A step voltage is then applied to the non-inverting input of the patch-clamp system. The output voltage variation of the patch-clamp system converter in response to the step voltage is then obtained and from that output voltage variation; the series resistance R_{E} of the electrode can be determined. After the series resistance is determined a sensor is connected between the second end of the electrode and ground. The steady state output of the patch-clamp system is then found and the sensor current is measured. The sensor series resistance R_{S} can then be determining from the measured sensor current i, the series resistance R_{E}, and the steady state output. Once the series resistance R_{E} is known, a predetermined voltage can be applied across the sensor by applying a compensated voltage to the non-inverting input, where the compensated voltage is equal to the predetermined voltage plus the sensor current i times the series resistance R_{S}.

In addition to compensating for resistances, the present invention can also be used to determine parasitic capacitances. To do so, after the sensor series resistance R_{S} has been determined the patch-clamp system is set up to produce a steady state response. A compensation step voltage is then applied to the non-inverting input of the patch-clamp system. The time constant of the output is then found. The input parasitic capacitance is then determined using the previously obtained sensor series resistance R_{S} and the time constant.

The principles of the present invention further enable new, useful, and non-obvious nanopore sequencers. Such a nanopore sequencer includes a nanopore sensor having an input resistance R_{N} and an input capacitance C_{N}. The nanopore sequencer further includes a patch-clamp circuit having a non-inverting input, an inverting input having a parasitic capacitance C_{P}, and an output. An electrode having an electrode series resistance R_{E} connects the nanopore sensor to the inverting input. A feedback resistor having a value R_{F} is connected between the output and the inverting input. The reset switch receives timing signals that cause the reset switch to selectively connect the output to the inverting input. A digital-to-analog circuit receives timed digital command voltages and applies stepped command voltages to the non-inverting input in response to the timed digital command voltages. The reset switch closes for a time T_{R} in synchronization with step changes in the stepped command voltages and then opens. T_{R} is selected to be sufficient to prevent saturation of the patch-clamp circuit without blanking out the stepped voltage. The stepped command voltages are selected to compensate for the nanopore resistance R_{N} and the electrode series resistance R_{E} so as to produce a predetermined voltage across the nanopore sensor.

The nanopore sensor may comprise a semi-conductive material or it may be a cell membrane. The patch-clamp circuit may include a current-to-voltage converter and a difference amplifier. The output is beneficially applied to an analog-to-digital converter that produces an amplified digital version of the current in the nanopore sensor. That amplified digital version can be input to a field programmable array and/or as an input to a computer. Preferably the computer operatively produces the timing signals and the timed digital command voltages.

### BRIEF DESCRIPTION OF THE DRAWINGS

The advantages and features of the present invention will become better understood with reference to the following detailed description and claims when taken in conjunction with the accompanying drawings, in which like elements are identified with like symbols, and in which:

FIG. 1(a) is a schematic depiction of a prior art resistive feedback patch-clamp circuit;

FIG. 1(b) is a depiction of a prior art capacitive feedback patch-clamp circuit;

FIG. 2 is a schematic depiction of a simplified compensated patch-clamp circuit in accord with the principles of the present invention;

FIG. 3(a) is a schematic depiction of the operation of the compensated patch-clamp circuit shown in FIG. 2 when reset switch 16 is closed;

FIG. 3(b) is a schematic depiction of the operation of the compensated patch-clamp circuit shown in FIG. 2 when reset switch 16 is open;

FIG. 4 is a schematic depiction of a compensated patch-clamp circuit in accord with the principles of the present invention that uses a digital-to-analog converter (DAC);

FIG. 5 illustrates a schematic depiction of a prior art patch-clamp system and a nanopore sensor;

FIG. 6 is a schematic depiction of a preferred embodiment compensated patch-clamp circuit;

FIG. 7 is a schematic depiction of a simplified version of the compensated patch-clamp circuit shown in FIG. 6 during early resistor compensation operations;

FIG. 8 is a schematic depiction of a simplified version of the compensated patch-clamp circuit shown in FIG. 6 during later resistor compensation operations;

FIG. 9 is an operational flow diagram for compensating nanopore sensor resistances;

FIG. 10 is an operational flow diagram for compensating nanopore sensor capacitances;

FIG. 11 is a schematic depiction of a simplified preferred embodiment compensated patch-clamp circuit during capacitor compensation; and

FIG. 12 is a schematic depiction of a simplified preferred embodiment compensated capacitor patch-clamp circuit.

FIG. 13 shows a three terminal nanopore sensor front end for practicing the present invention.

### DETAILED DESCRIPTION OF THE INVENTION

The presently disclosed subject matter now will be described more fully hereinafter with reference to the accompanying drawings in which one embodiment is shown. However, it should be understood that this invention may take many different forms and thus should not be construed as being limited to the embodiment set forth herein.

All publications mentioned herein are incorporated by reference for all purposes to the extent allowable by law. In addition, in the figures like numbers refer to like elements throughout. Additionally, the terms “a” and “an” as used herein do not denote a limitation of quantity, but rather denote the presence of at least one of the referenced items.

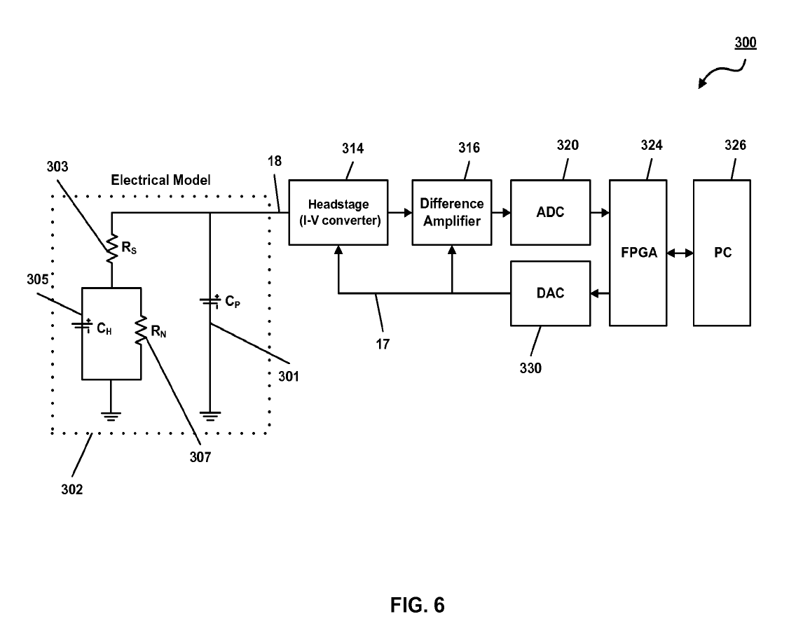

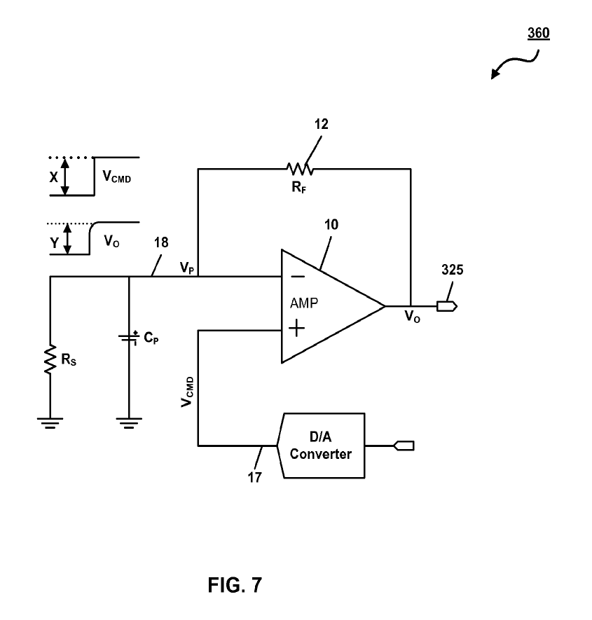

In what follows a generic nanopore sensor 302 (reference FIG. 6) is described, used, and compensated for. It should be understood that a nanopore sensor 302 might incorporate a living cellular membrane or it might incorporate a solid-state nanopore. Furthermore, while not all circuits that are subsequently described, specifically show a nanopore sensor 302, which is to better show the circuit operation, and thus it should be understood that a nanopore sensor 302 is, or can be, connected to the variously illustrated and described circuitry. Note also that where electrode series resistance is mentioned, sensor series resistance may sometimes be employed in some embodiments.

Devices suitable for use with the present invention are described in, for example, U.S. Pat. No. (U.S. Pat. No.) 5,795,782, U.S. Pat. No. 6,015,714, U.S. Pat. No. 6,267,872, U.S. Pat. No. 6,627,067, U.S. Pat. No. 6,746,594, U.S. Pat. No. 6,428,959, U.S. Pat. No. 6,617,113, and International Publication Number WO 2006/028508, each of which is hereby incorporated by reference in their entirety. Essentially while any individual device described herein may not be novel, the combination of the individual devices results in a new, useful, and non-obvious nanopore patch-clamp systems, DNA sequencers, and electrochemical applications for measuring biochemical analytic concentrations such as glucose, oxygen, neurotransmitters and pathogens that can be measured using transimpedance amplifiers or current-to-voltage converters.

Nanopore sensitivity, particularly in the case of solid-state nanopores, is determined by the pore size and the thickness. To identify a single nucleotide (≈0.35 nm) of single-stranded DNA in a nanopore sensor, the nanopore sensor will have a diameter of somewhere around 0.35 nm or less. That causes a nanopore capacitance of about:

$$C = {ɛ}_{r} {ɛ}_{0} \frac{A}{d} ,$$

where εᵣ, ε₀, A and d indicate a relative permittivity, the electric constant (8.854×10⁻¹² F m⁻¹), an exposed area, and thickness, respectively. Where atomic layers, i.e. Al₂O₃ and graphene, are used for the nanopore sensor the nanopore capacitance is larger, which results in longer dead-times (see below) when the command voltage changes. Such atomic layer sensors particularly benefit by the principles of the present invention.

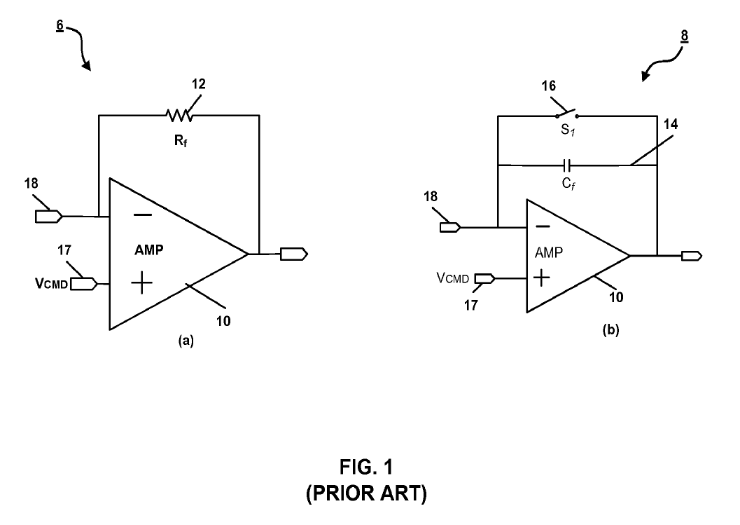

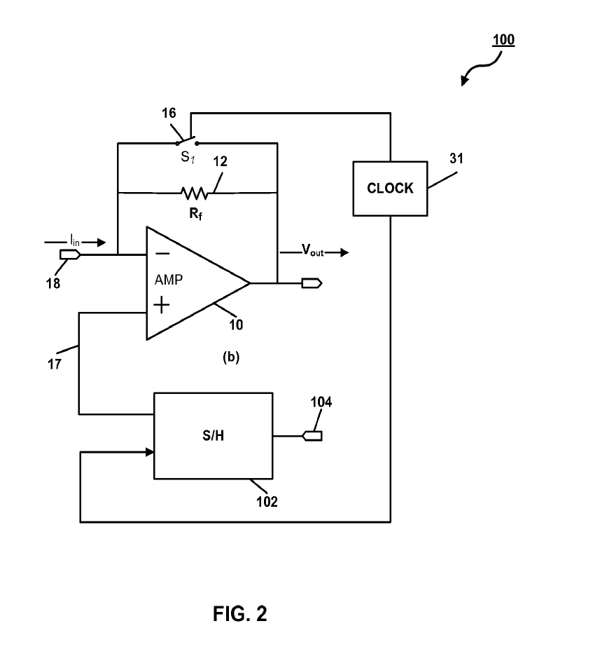

FIG. 2 illustrates a basic compensated patch-clamp circuit 100 that is in accord with the present invention. The basic compensated patch-clamp circuit 100 differs in hardware from the resistive feedback patch-clamp circuit 6 (see FIG. 1(a)) by the incorporation of a reset switch 16 for selectively shorting out the feedback resistor 12 and by the incorporation of a sample and hold circuit 102 that is disposed between the non-inverting input 18 and a command voltage V_{CMD} applied to the input 104 of the sample and hold circuit 102.

During operation the reset switch 16 is closed in synchronization with step transitions of the output of the sample and hold circuit 102. In practice those transitions and the reset switch 16 synchronization are controlled by timing pulses from a clock 31. For purposes of clarity of explanation those timing pulses and the clock 31 are left out of subsequent figures. However is should be understood that the reset switch 16 operates in synchronization with command voltage V_{CMD} changes, be they from a sample and hold circuit, a digital-to-analog converter, or some other circuit, and that some type of synchronized timing is required.

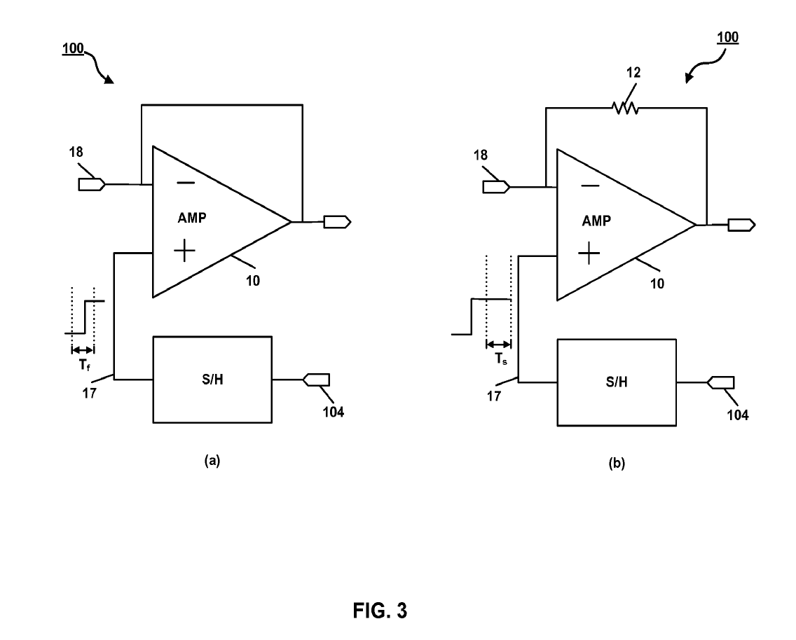

The basic compensated patch-clamp circuit 100 has two modes of operation: a transient mode when the command voltage W_{CMD} changes, depicted in FIG. 3(a), and a steady state mode when the command voltage V_{CMD} is stable, depicted in FIG. 3(b). In both operational modes it should be understood that the command voltage V_{CMD} has been digitized into discrete steps. During transient mode operation the saturation and associated dead-time of the op-amp 10 is avoided by closing the reset switch 16. The operation of the compensated patch-clamp circuit 100 is then similar to the capacitive feedback pulse clamp circuit shown in FIG. 1(b) and the op-amp 10 operates as a unity gain amplifier. In the steady-state mode the reset switch 16 is turned off and the basic compensated patch-clamp circuit 100 operates like the resistive-feedback patch-clamp shown in FIG. 1(a).

Because a feedback capacitor 14 is not used in the basic compensated patch-clamp circuit 100 periodic reset pulses are not required to remove built up charges. Furthermore, complex compensation circuitry is also not required because resistive-feedback is used. The basic compensated patch-clamp circuit 100 architecture enables the use of complex command voltage V_{CMD} waveforms and the use of various dwell times in addition to reduced hardware complexity.

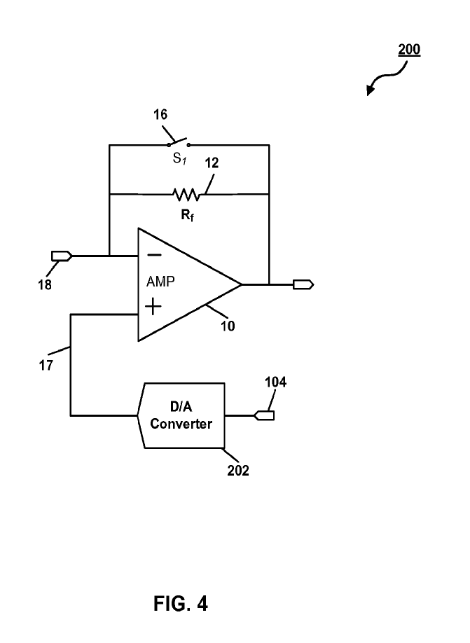

The basic compensated patch-clamp circuit 100 and its sample and hold circuit 102 represents a major change in nanopore patch-clamp circuits. One improvement to the basic compensated patch-clamp circuit 100 is shown in the improved compensated patch-clamp circuit 200 of FIG. 4. The improved compensated patch-clamp circuits 200 uses a low-pass filtered digital-to-analog converter 202 in place of the sample and hold circuit 102 shown in FIG. 2. The digital-to-analog converter 202 is an improvement because the digital-to-analog converter 202 can be directly connected to and controlled by a computerized system such as a personal computer. Such a computerized system is described subsequently; reference FIG. 6 and its supporting description. In addition, the reset switch 16 can be controlled either by a computer or by a field programmable gate array. However, timing synchronization of the reset switch 16 operations and command voltage V_{CMD} changes is still required, although the simple clock 31 shown in FIG. 2 may be replaced by clocked digital-to-analog converter 202 timing signals or timing derived from the output of the computer.

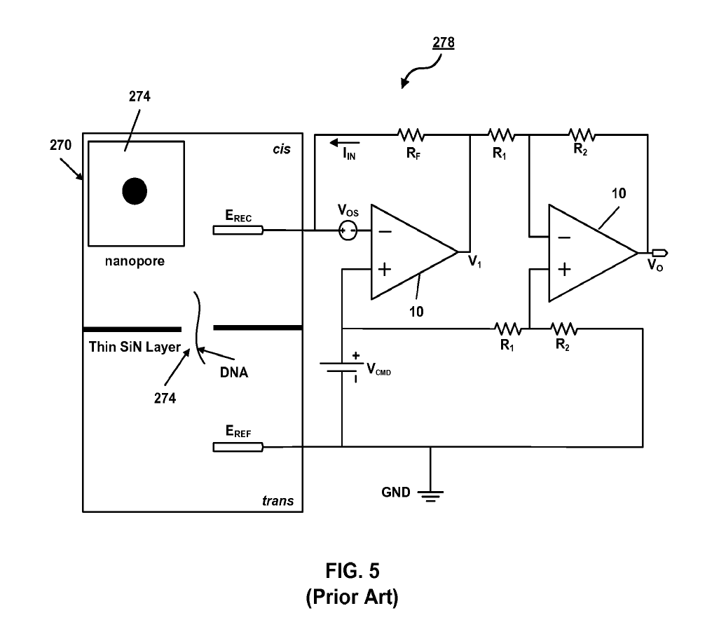

As noted patch-clamps have been used in prior art DNA sequencers. FIG. 5 shows a prior art DNA sequencer 270. It comprises a nanopore sensor 272 having two “channels”: a cis channel and a trans channel separated by a nanopore 274 through a semi-conductive material and retained in an ionic (KCl) fluid-filled container. The current that flows between the cis channel and the trans channel is converted by a first op-amp into a voltage (I-V conversion) and then amplified by difference amplifier. The basic patch-clamp amplifiers 6 and 8, reference FIG. 1, in practice are replaced by a two-stage patch-clamp amplifier 278 having an I-V conversion stage and a difference amplifier stage.

While the basic patch-clamp circuits 100 and 200 are by themselves new, beneficial and useful, the preferred embodiment of the present invention is the computerized compensated DNA sequencer 300 system shown in FIG. 6. The DNA sequencer 300 includes a nanopore sensor 302 which directly corresponds to the nanopore sensor 272 shown in FIG. 5 except that the nanopore sensor 302 may comprises cell membrane nanopore or a semi-conductive nanopore. For clarity of understanding FIG. 6 presents an electrical model of the nanopore sensor 302 with the understanding that its physical configuration will be that of the nanopore sensor 272 or its cell membrane counterpart. That electrical model includes a nanopore capacitance 304 (C_{N}), a nanopore resistance (R_{N}) 306, an electrode series resistance (R_{S}) 308, and an input parasitic capacitance (CO 310.

The nanopore sensor 302 is connected to the inverting input 18 of a patch-clamp circuit comprised of an input (I-V) converter 314 headstage and a difference amplifier 316, which is analogous to that shown in FIG. 5. The output of the patch-clamp circuit is input to an analog-to-digital converter 320 that digitizes its analog voltage input and applies its digitized output version as inputs to a field programmable gate array 324. The field programmable gate array 324 sends a suitably processed version of its received digitized voltage reading to a personal computer 326 (or another suitable computerized system).

The personal computer 326 performs data analysis on the nanopore sensor 302 reading. In addition, the personal computer PC 326 applies control signals to the field programmable gate array 324 which are subsequently used to control the operation of a digital-to-analog converter 330. The digital-to-analog converter 330 provides command voltages (V_{CMD}) to the non-inverting inputs 17 of the input (I-V) converter 314 headstage and the difference amplifier 316. Thus the operation of the DNA sequencer 300 is computer controlled, its outputs are available for data analysis, and patch-clamp compensation is provided as described below.

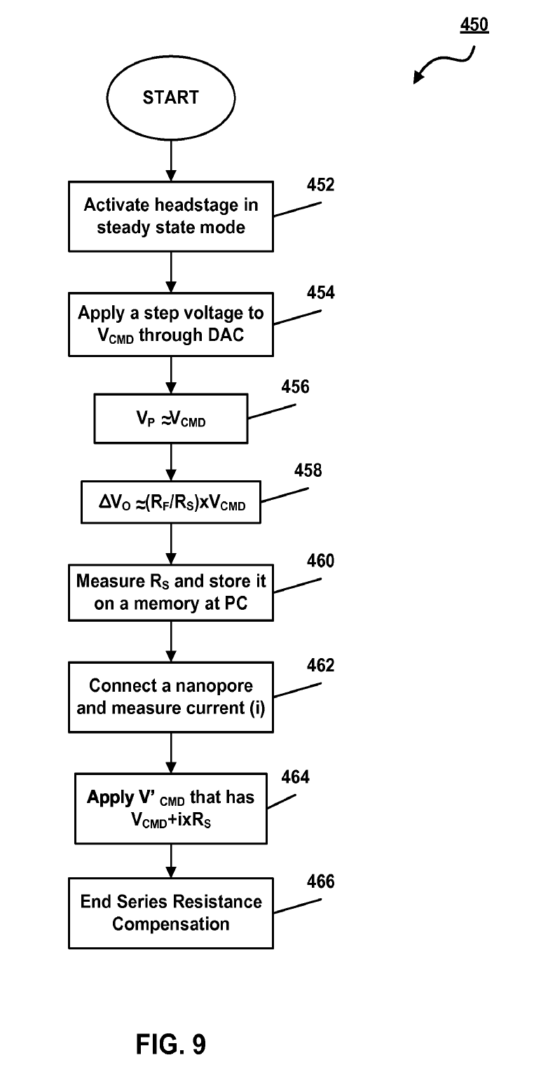

The DNA sequencer 300 is well suited for automated compensation. A compensation operation 450 is shown in the flow diagram of FIG. 9. That operation 450 starts and proceeds by activating the input (I-V) converter 314 headstage and the difference amplifier 316 in a steady state mode, step 452. Obtaining a steady state mode is explained with the aid of a simplified patch-clamp circuit 360 (the input (I-V) converter 314 headstage and the difference amplifier 316) shown in FIG. 6. Note that the simplified patch-clamp circuit 360 is shown without the nanopore sensor 302 and with the electrode series resistance (R_{S}) 308 and the input parasitic capacitance (C_{P}) 310 grounded. The series resistance (R_{S}) 308 and the parasitic capacitance (C_{P}) 310 are distributed and unavoidable. The command voltage (V_{CMD}) is set to a predetermined voltage (nominally ground). This causes the output voltage V_{O} on the output terminal 325 to become stable and the patch-clamp circuit 360 is placed in a steady-state mode. Note that in various embodiments the nanopore doesn't have the sensor series resistance.

After some time a V_{CMD} voltage step is applied, step 454 which, after some time delay, sets the voltage V_{P} across the series resistance (R_{S}) 308 and the parasitic capacitance (C_{P}) 310 to V_{CMD} see step 456. Next, the output voltage variation is measured, step 458. Note that the output voltage is digitized and applied to the PC 326. From the output voltage variation and from the known R_{F} 12 the value of the electrode series resistance R_{S} can be accurately measured (determined), step 460. The formula relating the output voltage variation and R_{S} is shown in step 458.

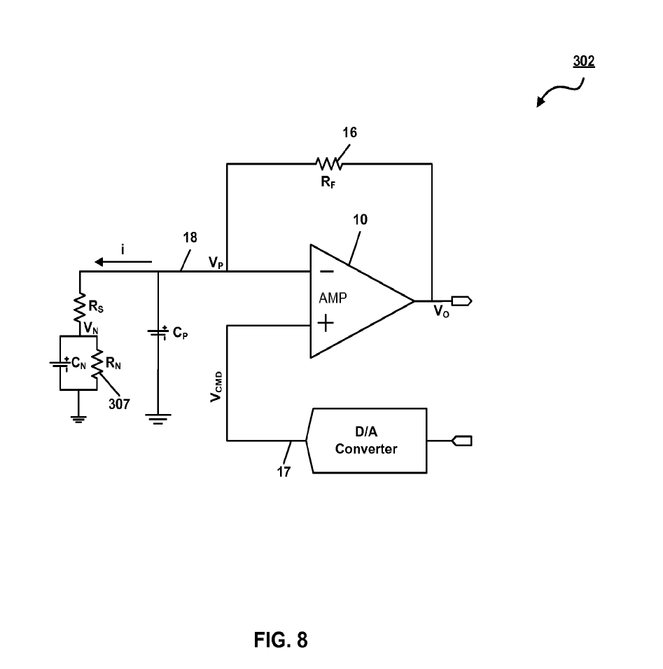

Next, a nanopore sensor 302 is applied to the patch-clamp amplifier 360 and the resulting nanopore current (i) is measured, step 462, reference FIG. 8. After the current (i) is measured the PC 326 causes the digital-to-analog converter 330 via the field programmable gate array 324 to generate another, different command voltage V′_{CMD} where V′_{CMD}=V_{CMD}+i×R_{S}, step 464. This is possible because R_{S} was previously found (steps 450 through 460). The nanopore sensor 302 resistance R_{N} 307 can also be determined from the output V_{O} variation. Series resistance compensation is ended, step 466. Since all nanopore related resistances have been determined the actual voltage applied across the nanopore sensor 302 can accurately be known despite the series resistance (R_{S}) 308, the parasitic capacitance (C_{P}) 310 and the nanopore resistance 307. Thus the nanopore sensor 302 resistive environment is accurately compensated for.

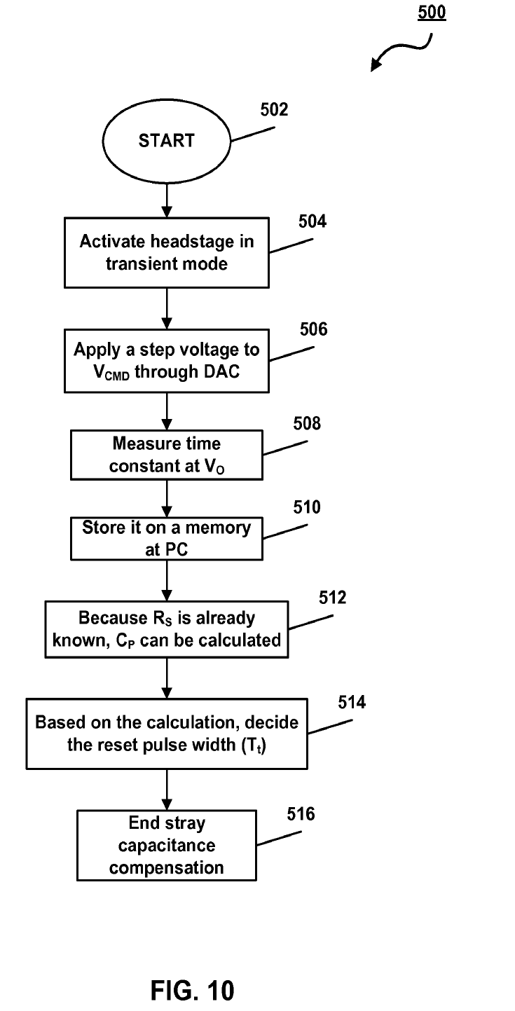

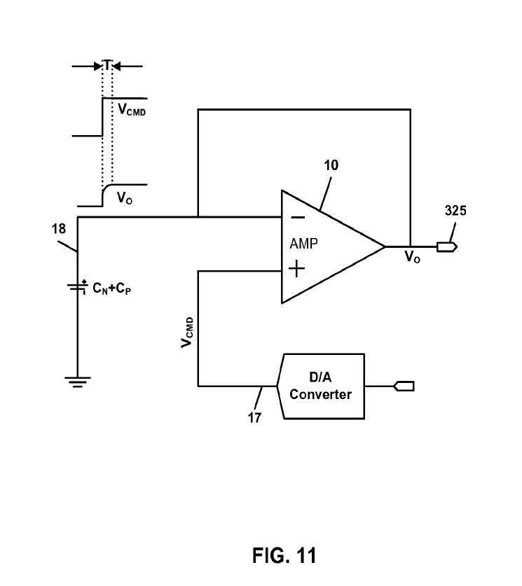

In addition to resistor compensation it is possible to compensate for capacitances. FIG. 10 illustrates the operation 500 of capacitance compensation. The operation 500 starts, step 502 and proceeds by entering a transient mode, step 504. FIG. 11 shows the transient mode which is entered by closing the reset switch 16 to short the inverting input to the output terminal 325, thus shorting out the feedback resistance R_{F} 12, (see FIG. 1(a)) and charging all capacitances. Next, a command voltage V_{CMD} step is applied, step 506. The output voltage V_{O} on the output terminal 325 is monitored and the time constant of V_{O} is measures, step 508, and stored in memory, step 510. Because the electrode series resistance R_{S} and the time constant have been determined the value of the parasitic capacitor C_{P}, which is much smaller than the nanopore electrode capacitance C_{N}, can be accurately calculated, step 512. From the calculated value of C_{P} a determination of an optimal reset pulse width (T) can be decided, step 514. The reset pulse width should be somewhat longer than the time constant found in step 506 but should not be so long as to blank out the voltage step. By blank out it is meant that the reset pulse width is so long that the response of the patch-clamp circuit to the voltage step cannot be determined by the system before another step occurs. That reset pulse width delay compensates for the input parasitic capacitances including the inverting input electrode, the connecting cable, and the nanopore sensor and capacitor compensation ends, step 516.

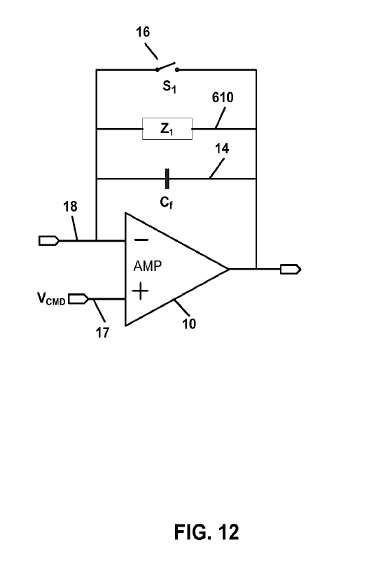

While the foregoing has described a novel resistive feedback patch-clamp system, its use in DNA sequencing, and automated compensation based on a resistive patch-clamp circuit, the principles of the present invention are also useful to capacitive patch-clamp circuits. FIG. 12 helps illustrate how the compensation technique of the present invention can be applied to the capacitive-feedback transimpedance amplifiers. Periodic reset pulses are not required because of the high impedance Z₁ 610 caused by unavoidable leakage. By eliminating the periodic resets the glitch at the input due to charge and clock feed-through are avoided. However, Z₁ still requires compensation as does the parasitic input capacitance C_{P} and the electrode series resistance R_{S}. By adding the reset switch 16 in parallel with Z₁ and C_{f} 14 the compensation procedure for the capacitive-feedback TIA are the same as previously described.

Additional embodiments and disclosures are as follows.

The invention herein disclosed provides for devices and methods that can detect and quantify individual nucleotides in a polynucleotide. The device can be a solid-state nanopore or a nanopore positioned at a defined site, for example, upon a substrate and/or surface.

The devices herein disclosed may be used in many applications, including, but not limited to, a nanopore system. The system can avoid ‘dead-time’ by placing a switch to a conventional transimpedance amplifier with a resistive feedback. Various discrete waveforms may be generated and applied to the voltage command by using a sample/hold circuit or DAC for the command voltage control. The voltage patch-clamp amplifier can be fully controlled by a computer interface system.

The invention also discloses for a method of compensating for the feedback resistors as disclosed above. The invention further discloses a method for compensating for the probe input capacitance.

The invention can be used to detect the position and measure the quantity of a molecule relative to the defined site. In one example, the defined site is a nanopore. The molecule can be positioned by varying the potential difference on either side of the nanopore. The molecule can be a macromolecule and can further comprise a polyion, such as a polyanion and/or a polycation. In one a preferred embodiment, the polyion is a polynucleotide. In another preferred embodiment the polyion is a polypeptide. The substrate and/or surface can delimit two chambers and can further comprise a pore, the pore located at the substrate or surface. One of the chambers is cis to the pore and the other chamber is trans to the pore. The molecule can be positioned by varying the potential difference between the chambers. Preferably, the molecule is initially present in the cis chamber. The presence and/or absence and/or change in the molecular composition can be detected by measuring the electric current through the pore. The invention can be used as a sensor that detects molecules. The invention is of particular use in the fields of molecular biology, structural biology, cell biology, molecular switches, molecular circuits, and molecular computational devices, and the manufacture thereof.

The invention provides devices and methods for using the same. The devices may be used in a nanopore device system or another suitable system. In one exemplary embodiment, the device is a voltage patch-clamp circuit, comprising: a clock producing clock signals having clock transitions; a differential amplifier having a non-inverting input, an inverting input, and an output; a feedback resistor connected between said output and said inverting input; a reset switch receiving said clock signals, said reset switch for selectively connecting said output to said inverting input in response to clock signals; and a sample and hold circuit receiving clock signals and command voltages, said sample and hold circuit for digitizing command voltages in response to clock signals and for applying digitized command voltages to said non-inverting input; wherein said reset switch is closed during a clock transition to reduce the gain of said differential amplifier; and wherein said reset switch is opened after said clock transition to increase the gain of said differential amplifier.

In another exemplary embodiment, the system can be used for a method of amplifying small current variations in a sensor, comprising the steps of: digitizing command voltages in accord with clock signals; applying voltages derived from digitized command voltages to a sensor to induce variations in the sensor current; amplifying the variations in sensor current to produce an output; reducing the amplification applied to the variations in sensor current when clock signals change so as to limit saturation; and increasing the amplification applied to the variations in sensor current when clock signals are not changing.

In yet another exemplary embodiment the system can be used for a method of compensating the series resistance of a nanopore sensor, comprising the steps of: activating a current-to-voltage converter to achieve a steady state response; applying a step voltage to a non-inverting input of the current-to-voltage converter such that the resulting voltage applied to inverting input of the current-to-voltage converter is substantially equal to the step voltage; determining the output voltage variation of the current-to-voltage converter to the step voltage; measuring the series resistance of a nanopore sensor; connecting the nanopore sensor to the non-inverting input of the current-to-voltage converter; measuring the nanopore sensor current; and compensating the nanopore sensor by applying a voltage to the inverting input of the current-to-voltage converter equal to the step voltage plus the nanopore sensor current times the series resistance.

In a further embodiment, the system can be used for a method of compensating the series resistance of a cell membrane sensor, comprising the steps of: activating a current-to-voltage converter to achieve a steady state response; applying a step voltage to a non-inverting input of the current-to-voltage converter such that the resulting voltage applied to inverting input of the current-to-voltage converter is substantially equal to the step voltage; determining the output voltage variation of the current-to-voltage converter to the step voltage; measuring the series resistance of a cell membrane sensor; connecting the cell membrane sensor to the non-inverting input of the current-to-voltage converter; measuring the cell membrane sensor current; and compensating the cell membrane sensor by applying a voltage to the inverting input of the current-to-voltage converter equal to the step voltage plus the cell membrane sensor current times the series resistance.

Additionally, The system can also be used for a method of compensating for the input parasitic capacitance of a nanopore sensor, comprising the steps of: connecting a nanopore sensor to the non-inverting input of a current-to-voltage converter; obtaining the series resistance of the nanopore sensor; activating the current-to-voltage converter to achieve a steady state response; applying a step voltage to a non-inverting input of the current-to-voltage converter; determining the time constant of the current-to-voltage converter to the step voltage; and determining the input parasitic capacitance of the nanopore sensor from the series resistance of a nanopore sensor and the determined time constant.

In an alternative embodiment, the system can be used for a method of compensating for the input parasitic capacitance of a cell membrane sensor, comprising the steps of: connecting a cell membrane sensor to the non-inverting input of a current-to-voltage converter; obtaining the series resistance of the cell membrane sensor; activating the current-to-voltage converter to achieve a steady state response; applying a step voltage to a non-inverting input of the current-to-voltage converter; determining the time constant of the current-to-voltage converter to the step voltage; and determining the input parasitic capacitance of the cell membrane sensor from the series resistance of a cell membrane sensor and the determined time constant.

The nanopore device systems may comprise ‘cis’ and ‘trans’ chambers connected by an electrical communication means. In one embodiment the chambers comprise a medium, the medium selected from the group consisting of an aqueous medium, a non-aqueous medium, an organic medium, or the like. In one embodiment the medium is a fluid. In an alternative embodiment the medium is a gas. In one embodiment the electrical communication means is a solid state pore comprising, for example, silicon nitride, bifunctional alkyl sulfide, and/or gold or other metal or alloy. In the alternative, the cis and trans chambers are separated by a thin film comprising at least one pore or channel. In one preferred embodiment, the thin film comprises a a compound having a hydrophobic domain and a hydrophilic domain. In a more preferred embodiment, the thin film comprises a a phospholipid. The devices further comprise a means for applying an electric field between the cis and the trans chambers. In one embodiment the pore or channel accommodates a part of the polyion. In another embodiment the pore or channel accommodates a part of the molecule. In one preferred embodiment, the molecule is a macromolecule. In another preferred embodiment the polyion is selected from the group consisting of polynucleotides, polypeptides, phospholipids, polysaccharides, and polyketides.

In one embodiment the compound comprises a an enzyme. The enzyme activity can be, for example, but not limited to, enzyme activity of proteases, kinases, phosphatases, hydrolases, oxidoreductases, isomerases, transferases, methylases, acetylases, ligases, lyases, ribozyme, and the like. In a more preferred embodiment the enzyme activity can be enzyme activity of DNA polymerase, RNA polymerase, endonuclease, exonuclease, DNA ligase, DNase, uracil-DNA glycosidase, kinase, phosphatase, methylase, acetylase, glucose oxidase, ribozyme, and the like.

In still a further interesting embodiment, the pore is sized and shaped to allow passage of an activator, wherein the activator is selected from the group consisting of ATP, NAD⁺, NADP⁺, diacylglycerol, phosphatidylserine, eicosinoids, retinoic acid, calciferol, ascorbic acid, neuropeptides, enkephalins, endorphins, 4-aminobutyrate (GABA), 5-hydroxytryptamine (5-HT), catecholamines, acetyl CoA, S-adenosylmethionine, hexose sugars, pentose sugars, phospholipids, lipids, glycosyl phosphatidyl inositols (GPIs), and any other biological activator.

In certain embodiments the pore is sized and shaped to allow passage of a monomer, wherein the monomer is selected from the group consisting of dATP, dGTP, dCTP, dTTP, UTP, alanine, cysteine, aspartic acid, glutamic acid, phenylalanine, glycine, histidine, isoleucine, lysine, leucine, methionine, asparagines, proline, glutamine, arginine, serine, threonine, valine, tryptophan, tyrosine, hexose sugars, pentose sugars, phospholipids, lipds, and any other biological monomer.

In yet another embodiment the pore is sized and shaped to allow passage of a cofactor, wherein the cofactor is selected from the group consisting of Mg²⁺, Mn²⁺, Ca²⁺, ATP, NAD⁺, NADP⁺, and any other biological cofactor.

In one important embodiment, the pore or channel comprises a a biological molecule, or a synthetic modified or altered biological molecule. Such biological molecules are, for example, but not limited to, an ion channel, such as a-hemolysin, a nucleoside channel, a peptide channel, a sugar transporter, a synaptic channel, a transmembrane receptor, such as GPCRs, a receptor tyrosine kinase, and the like, a T-cell receptor, an MHC receptor, a nuclear receptor, such as a steroid hormone receptor, a nuclear pore, or the like.

In an alternative, the compound comprises a non-enzyme biological activity. The compound having non-enzyme biological activity can be, for example, but not limited to, proteins, peptides, antibodies, antigens, nucleic acids, peptide nucleic acids (PNAs), locked nucleic acids (LNAs), morpholinos, sugars, lipids, glycosyl phosphatidyl inositols, glycophosphoinositols, lipopolysaccharides, or the like. The compound can have antigenic activity. The compound can have ribozyme activity. The compound can have selective binding properties whereby the polymer binds to the compound under a particular controlled environmental condition, but not when the environmental conditions are changed. Such conditions can be, for example, but not limited to, change in [H⁺], change in environmental temperature, change in stringency, change in hydrophobicity, change in hydrophilicity, or the like.

In one embodiment the macromolecule comprises a enzyme activity. The enzyme activity can be, for example, but not limited to, enzyme activity of proteases, kinases, phosphatases, hydrolases, oxidoreductases, isomerases, transferases, methylases, acetylases, ligases, lyases, and the like. In a more preferred embodiment the enzyme activity can be enzyme activity of DNA polymerase, RNA polymerase, endonuclease, exonuclease, DNA ligase, DNase, uracil-DNA glycosidase, kinase, phosphatase, methylase, acetylase, glucose oxidase, or the like. In an alternative embodiment, the macromolecule can comprise more that one enzyme activity, for example, the enzyme activity of a cytochrome P450 enzyme. In another alternative embodiment, the macromolecule can comprise more than one type of enzyme activity, for example, mammalian fatty acid synthase. In another embodiment the macromolecule comprises a ribozyme activity.

In another embodiment, the invention provides a compound, wherein the compound further comprises a linker molecule, the linker molecule selected from the group consisting of a thiol group, a sulfide group, a phosphate group, a sulfate group, a cyano group, a piperidine group, an Fmoc group, and a Boc group. In another embodiment the compound is selected from the group consisting of a bifunctional alkyl sulfide and gold.

Devices that can be used to carry out the methods of the instant invention are described in for example, U.S. Pat. No. (U.S. Pat. No.) 5,795,782, U.S. Pat. No. 6,015,714, U.S. Pat. No. 6,267,872, U.S. Pat. No. 6,627,067, U.S. Pat. No. 6,746,594, U.S. Pat. No. 6,428,959, U.S. Pat. No. 6,617,113, and International Publication Number WO 2006/028508, each of which is hereby incorporated by reference in their entirety.

While the forgoing has described the inventive compensation technique in terms of patch-clamps it can also be employed in applications where excessive dead-times must be avoided. Accurate reset pulse widths can reduce the dead-times.

It is to be understood that while the figures and the above description illustrates the present invention, they are exemplary only. They are not intended to be exhaustive or to limit the invention to the precise forms disclosed, and obviously many modifications and variations are possible in light of the above teaching. Others who are skilled in the applicable arts will recognize numerous modifications and adaptations of the illustrated embodiments that remain within the principles of the present invention. Therefore, the present invention is to be limited only by the appended claims.

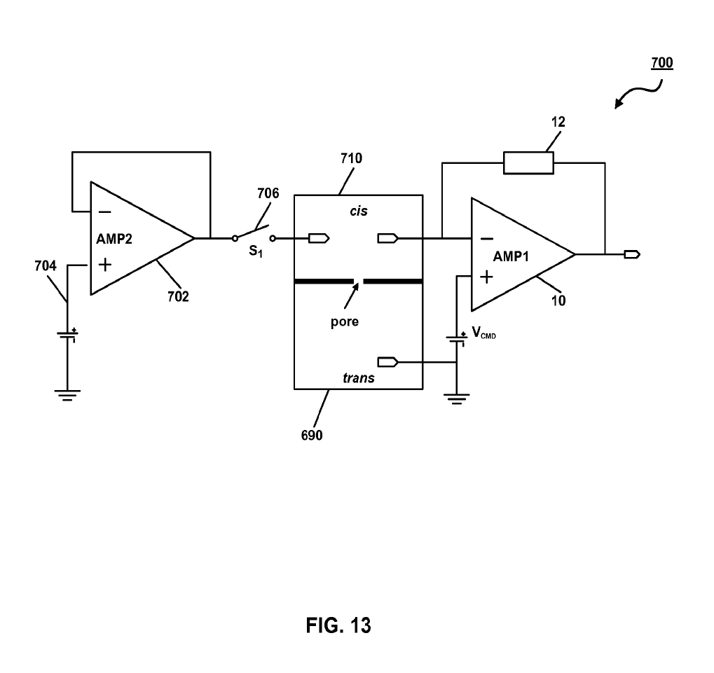

While the foregoing has explained the present invention using traditional two-electrode nanopore sensors the principles of the present invention are flexible enough to be used with other architectures. For example, FIG. 13 shows a three electrode nanopore sensor 690 front end circuit 700. A unity-gain buffer amplifier 702 buffers the command voltage VCMD on its non-inverting input. Its buffered output is connected to the cis chamber through a switch S1 706. When the command voltage VCMD is changed the switch S1 706 turns on to inject current to charge the nanopore sensor's capacitance CN until the cis chamber 710 potential equals VCMD. This assists compensating for dead times. The compensation technique invented here can be applied to nanopore application, patch-clamp application and electrochemical applications to measure biochemical analytic concentrations, such as glucose, oxygen, neurotransmitters and pathogens that can be measured using a transimpedance amplifier or a current-to-voltage converter.

1. A patch-clamp system, comprising:
a circuit producing timing signals;
a differential amplifier circuit having a non-inverting input, an inverting input with a parasitic capacitance and connected to an electrode resistance, and an output;
a feedback resistor connected between said output and said inverting input;
a reset switch receiving said timing signals, said reset switch for selectively connecting said output to said inverting input in response to said timing signals;
a command voltage circuit receiving timing signals and command voltages, said command voltage circuit for applying stepped command voltages to said non-inverting input in response to said timing signals; and
a sensor having an input capacitance and a series resistance, said sensor operatively connected to said inverting input;
wherein said reset switch is closed in synchronization with a step change in said stepped command voltages for a time TR;
wherein said reset switch is opened after said time TR;
wherein said time TR is sufficient to prevent saturation of said differential amplifier circuit without blanking said stepped voltage; and
wherein said stepped command voltages are selected to compensated for said series resistance and said electrode resistance to produce a predetermined voltage across said sensor.

2. The patch-clamp system according to claim 1, wherein said sensor comprises a nanopore sensor.

3. The patch-clamp system according to claim 1, wherein said differential amplifier circuit includes a current-to-voltage converter and a difference amplifier.

4. The patch-clamp system according to claim 1, wherein said command voltage circuit comprises a sample and hold circuit.

5. The patch-clamp system according to claim 1, wherein said command voltage circuit comprises a digital-to-analog converter.

6. The patch-clamp system according to claim 5, wherein said output is applied to an analog-to-digital converter that produces an amplified digital version of said current in said sensor.

7. The patch-clamp system according to claim 6, wherein said amplified digital version is applied to a field programmable array.

8. The patch-clamp system according to claim 6, wherein said amplified digital version is input to a computer.

9. The patch-clamp system according to claim 8, wherein said computer causes said command voltages to be applied to said command voltage circuit.

10. A method of compensating a sensor in a patch-clamp system, comprising the steps of:
a) connecting a first end of an electrode to an inverting input of a patch-clamp system;
b) connecting the second end of the electrode to ground;
c) connecting a feedback resistor RF between the inverting input and the output of the patch-clamp system;
d) obtaining a steady state output of the patch-clamp system by setting the voltage on the non-inverting input to a reference voltage;
e) applying a step voltage to the non-inverting input;
f) determining the output voltage variation of the patch-clamp system converter in response to the step voltage;
g) calculating the electrode series resistance RE using the output voltage variation determined in step f);
h) connecting a sensor between the second end of the electrode and ground;
i) obtaining a steady state output of the patch-clamp system by setting the non-inverting input to a reference voltage;
j) measuring the sensor current i after the steady state output is achieved;
k) determining the sensor series resistance RS from the measured sensor current i, the electrode series resistance RE, and the steady state output;
l) using the sensor by obtaining a predetermined voltage across the sensor by applying a compensated voltage to the non-inverting input where the compensated voltage is equal to the predetermined voltage plus the sensor current i times the sensor series resistance RS.

11. The method of compensating a sensor in a patch-clamp system according to claim 10, further including the steps:
activating the patch-clamp system to achieve a steady state response after the sensor series resistance RS has been determined;
applying a compensation step voltage to a non-inverting input of the patch-clamp system;
determining the time constant of the output of the patch-clamp system to the compensation step voltage; and
determining the input parasitic capacitance of the sensor from the sensor series resistance RS and the determined time constant.

12. The method of compensating a sensor in a patch-clamp system according to claim 11, further including the step of determining a reset pulse width based on the determined input parasitic capacitance.

13. A nanopore sequencer, comprising:
a nanopore sensor having an input resistance RN and an input capacitance CN;
a patch-clamp circuit having a non-inverting input, an inverting input with a parasitic capacitance CP, and an output;
an electrode connecting said nanopore sensor to said inverting input, said electrode having an electrode series resistance RE;
a feedback resistor connected between said output and said inverting input;
a reset switch receiving timing signals, said reset switch for selectively connecting said output to said inverting input in response to said timing signals;
a digital-to-analog circuit receiving timed digital command voltages, said digital-to-analog circuit for applying stepped command voltages to said non-inverting input in response to said timed digital command voltages; and
wherein said reset switch is closed in synchronization with a step change in said stepped command voltages for a time TR;
wherein said reset switch is opened after said time TR;
wherein said time TR is sufficient to prevent saturation of said patch-clamp circuit without blanking said stepped voltage; and
wherein said stepped command voltages are selected to compensate for said input resistance RN and said electrode series resistance RE so as to produce a predetermined voltage across said nanopore sensor.

14. The nanopore sequencer according to claim 13, wherein said nanopore sensor comprises a semi-conductive material.

15. The nanopore sequencer according to claim 13, wherein said nanopore sensor comprises a cell membrane.

16. The nanopore sequencer according to claim 13, wherein said 1, wherein said patch-clamp circuit includes a current-to-voltage converter and a difference amplifier.

17. The nanopore sequencer according to claim 13, wherein said output is applied to an Analog-to-Digital converter that produces an amplified digital version of said current in said nanopore sensor.

18. The nanopore sequencer according to claim 17, wherein, wherein said amplified digital version is input to a field programmable array.

19. The nanopore sequencer according to claim 17, wherein said amplified digital version is input to a computer.

20. The nanopore sequencer according to claim 13, wherein said computer operatively produces said timing signals and said timed digital command voltages.

21. The nanopore sequencer according to claim 13 adapted to sequence a polynucleotide.
